(19) United States
(12) Patent Application Publication
(10) Pub. No.: US 2024/0416038 A1
(43) Pub. Date: Dec. 19, 2024

(54) APPARATUS AND METHOD FOR FILLING SYRINGES

(71) Applicant: DOW SILICONES CORPORATION, Midland, MI (US)

(72) Inventor: Jinho KIM, Jincheon-gun, Chungcheongbuk-do (KR)

(21) Appl. No.: 18/580,574

(22) PCT Filed: Jun. 15, 2022
(86) PCT No.: PCT/KR2022/008464

§ 371 Date: Jan. 18, 2024

(30) Foreign Application Priority Data
Jul. 22, 2021 (KR) 10-2021-0096167

(51) Int. Cl. A61M 5/20; A61M 5/31

(52) U.S. Cl. CPC A61M 5/2033; A61M 5/3129

(57) ABSTRACT

The present disclosure relates to an apparatus for filling a syringe. The apparatus comprises: a pipeline for delivering a low viscous material discharged from a container; at least one downwardly directed injection port to connect with a syringe to be filled with the material delivered through the pipeline; a piston disposed within the syringe to be linearly movable in a longitudinal direction of the syringe and sealing the injected material between the injection port and the piston; and a syringe rod coupled to the syringe so as to press the piston into the syringe and to indicate a position of the piston. Each injection port has a needle valve to open and close the injection port.

### DESCRIPTION

### Technical Field

The present invention relates to an apparatus for filling a syringe with a low viscus material, and a method using such an apparatus.

### Background Art

Syringes for containing flowable materials are formed with a dispensing nozzle at one end and a larger aperture at the other end, wherein the dispensing nozzle can be blocked or sealed, and the larger aperture can be hermetically sealed by a piston. To discharge the materials within the syringes, the dispensing nozzle is either unblocked, or the seal broken, and the piston is pushed into the syringe displacing the material within the syringe through the dispensing nozzle.

In order to manufacture the flowable material-filled syringes, apparatuses for filling syringes or cartridges by means of a high-pressure pumping system are known.

For example, Patent Document 1 discloses a liquid filling apparatus comprising: a liquid pressing valve, a syringe, and a cylinder with a cylinder rod, wherein the liquid pressing valve presses liquid supplied from a tank, a pump, and a cartridge to discharge to the syringe, wherein the syringe can be filled with the liquid discharged from the liquid pressing valve at a constant capacity, and wherein a cylinder rod in the cylinder presses the liquid charging inside the syringe at a constant pressure when the fluid discharged from the liquid pressing valve is lifted inside the syringe in a state where the cylinder rod is inserted in a lower end of the syringe.

Patent Document 2 discloses a hydraulic liquid charging apparatus comprising: a base body including a fluid storage tank and a hydraulic pressure supply motor; a liquid supply unit; and a liquid charging unit, wherein the liquid supply unit comprises: left and right hydraulic cylinders; a liquid storage container; a piston connection rod; and a pressing piston, and wherein the liquid charging unit comprises: a dispenser valve; a syringe; and an emission preventing cylinder having a cylinder rod and supports an internal cap of the syringe.

Patent Document 3 discloses a liquid syringe filling apparatus comprising: a storage container sealed by a cover to store a liquid to be pumped; a first pressurizing unit to push the liquid inside the storage container to the outside; a pumping valve connected to the cover through a liquid inflow pipe to have a syringe mounted to allow the liquid pumped to fill the inside of the syringe; and a controller which controls a pressurizing force of the first pressurizing unit and a pressure of air supplied to the pumping valve and to control a pumping time difference for replacing the syringe, wherein the cover includes an air vent installed on the cover to discharge air bubbles contained in the liquid stored in the storage container.

Patent Document 4 discloses a liquid syringe filling apparatus comprising: a storage container hermetically sealed by a ram plate; wherein an outlet is located on the container or the ram plate; a pipeline to deliver the material discharged from the outlet by means of pressing with the ram plate; a syringe connected to an injection port with the material delivered through the pipeline, wherein a piston disposed in the syringe to be linearly movable in a longitudinal direction of the syringe and scaling the injected material between the injection port and the piston, wherein a sensor rod coupled to the syringe so as to be movable between a spaced apart position spaced from the piston and a contact position in which the piston is pressed against the piston by a weight of the sensor rod, and wherein the injection port has a needle valve to open and close the injection port; and then closing the injection port to stop injecting the material.

However, these apparatuses have the problem that, when the flowable material has low viscosity, air bubble generates in the flowable material with the syringe cause of that the material is easy to leak out of the syringe filling nozzle.

### PRIOR ART DOCUMENTS

### Patent Documents

Patent Document 1: Korean Patent Application Publication No. 10-2012-0058492 A

Patent Document 2: Korean Patent Application Publication No. 10-2017-0125702 A

Patent Document 3: Korean Patent Application Publication No. 10-2019-0063236 A

Patent Document 4: Korean Utility Model Application Publication No. 20-2021-0000786 U

### DISCLOSURE OF INVENTION

### Technical Problem

It is an object of the present invention to provide an apparatus for filling a syringe enable rapid filling a low viscus material, and prevents bubbles forming and the material leaking. In addition, it is another object of the present invention to provide a method for filling a syringe enable rapid filling a low viscus material, and prevents bubbles forming and the material leaking by using the apparatus.

### Solution to Problem

The apparatus for filling a syringe of the present invention, comprises:

a pipeline for delivering a low viscous material discharged from a container;

at least one downwardly directed injection port to connect with a syringe to be filled with the material delivered through the pipeline;

a piston disposed within the syringe to be linearly movable in a longitudinal direction of the syringe and sealing the injected material between the injection port and the piston; and

a syringe rod coupled to the syringe so as to press the piston into the syringe and to indicate a position of the piston,

wherein each injection port has a needle valve to open and close the injection port.

In various embodiments, the injection port has a syringe filling nozzle.

In various embodiments, the pipeline is equipped with a mesh screen filter.

In various embodiments, the pipeline is disassembled for cleaning.

In various embodiments, the syringe rod is composed of a syringe rod, a spring to press the rod, and a syringe rod barrel to set a lower end of the spring and to expose an upper end of the rod.

In various embodiments, the syringe rod is composed of a syringe rod, and a limit sensor mounted on a head of the syringe rod.

In various embodiments, the injection port is opened or closed by the position of the piston.

The method for filling a syringe of the present invention, comprises the following steps:

delivering a low viscous material discharged from a container through a pipeline;

filling a syringe connected to a downwardly directed injection port with the material delivered through the pipeline, wherein a piston is disposed within the syringe to be linearly movable in a longitudinal direction of the syringe and sealing the injected material between the injection port and the piston, wherein a syringe rod is coupled to the syringe so as to press the piston into the syringe and indicates a position of the piston; and then

closing the injection port to stop injecting the material by means of a needle valve of the injection port.

In various embodiments, the injection port is opened or closed by the position of the piston.

### Advantageous Effects of Invention

The apparatus and method for filling a syringe of the present invention enable rapid filling a low viscus material, and prevents bubbles forming and the material leaking.

### BRIEF DESCRIPTION OF DRAWINGS

FIG. 1 is an overall side view of the present apparatus.

FIG. 2 is a side view of a pipeline with a mesh screen filter according to the present apparatus.

FIG. 3 is a side view showing that a pipeline according to the present apparatus is dis-assemble.

FIG. 4 is a schematic view showing needle valve of the injection port according to the present apparatus.

FIG. 5 is a schematic view showing syringe filling nozzle of the injection port according to the present apparatus.

FIG. 6 is a schematic view showing a process to fix a syringe on an injection port according to the present apparatus.

FIG. 7 is a schematic view showing a process to file a low viscus material into a syringe according to the present apparatus.

FIG. 8 is a schematic view showing a syringe rod according to the present apparatus.

FIG. 9 is a schematic view showing another syringe rod according to the present apparatus.

FIG. 10 is another overall side view of the present apparatus.

FIG. 11 is a side view showing the present apparatus with multi-injection ports.

FIG. 12 is another side view showing the present apparatus with multi-injection ports.

### MODE FOR THE INVENTION

### Definitions

The terms “comprising” or “comprise” are used herein in their broadest sense to mean and encompass the notions of “including,” “include,” “consist(ing) essentially of,” and “consist(ing) of.” The use of “for example,” “e.g.,” “such as,” and “including” to list illustrative examples does not limit to only the listed examples. Thus, “for example” or “such as” means “for example, but not limited to” or “such as, but not limited to” and encompasses other similar or equivalent examples.

The terms “syringe” or “syringes” are used herein in their broadest sense to mean and encompass the notions of “cylindrical container formed with a dispensing nozzle at one end and a larger aperture at the other end,” such as “cartridge.” The terms “piston” or “pistons” are used herein in their broadest sense to mean and encompass the notions of “materials to hermetically seal the larger aperture of the syringes or cartridges,” such as “plunger.” To discharge the materials of the syringes, the dispensing nozzle is either unblocked, or the seal broken, and the piston is pushed into the syringe displacing the material in the syringe through the dispensing nozzle.

Hereinafter, exemplary embodiments of the present apparatus and method will be described in detail. The exemplary embodiments serve as examples embodied for describing the present apparatus and method without being limited to the scope of the present apparatus and method.

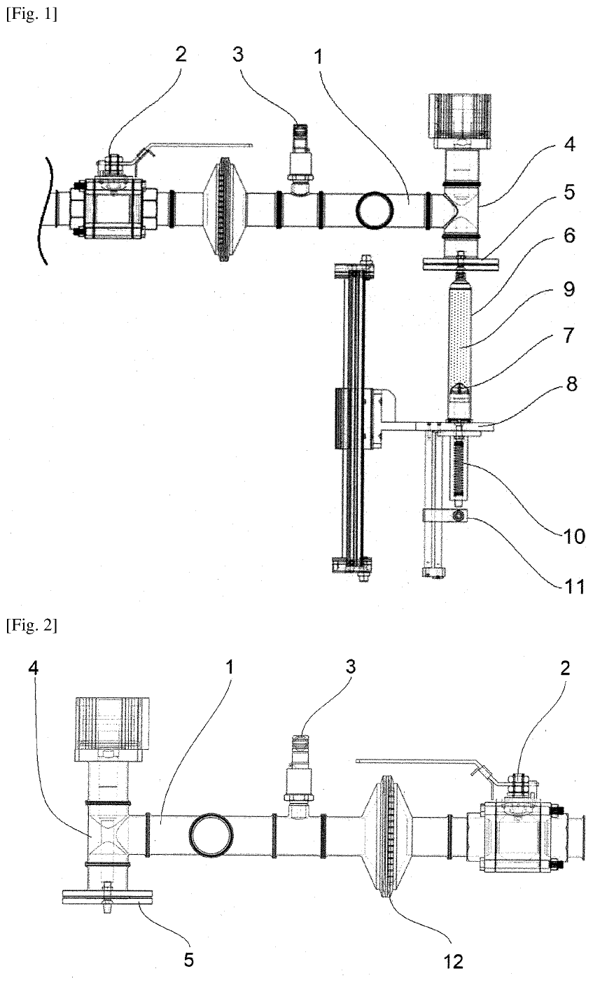

FIG. 1 shows an overall side view of the apparatus for filling a syringe of the present invention, and an apparatus comprising:

a low viscus material-delivering part containing: a pipeline (1), and optionally a valve (2) and a pressure sensor (3); and

a low viscus material-injecting part containing: a needle valve (4), an injection port (5), a syringe (6), a piston (7) and a syringe fixing plate (8).

The low viscus material is discharged in a container (not shown) and is delivered through the pipeline (1). The low viscus material is not limited, but it is exemplified by moisture-curable materials, UV-curable materials, and heat-curable materials, among these, moisture-curable materials are preferable. A viscosity at 25°° C. of the low viscus material is not limited, but it is preferably not more than 1,000 mPa·s.

In the embodiment shown in FIG. 1, the valve (2) and the pressure sensor (3) are presented, but they are optional. A pressure of the low viscus material can be controlled by the valve (2), the pressure sensor (3), and a control panel (not shown). The pressure can be changed according to the low viscus material and required syringe filling speed.

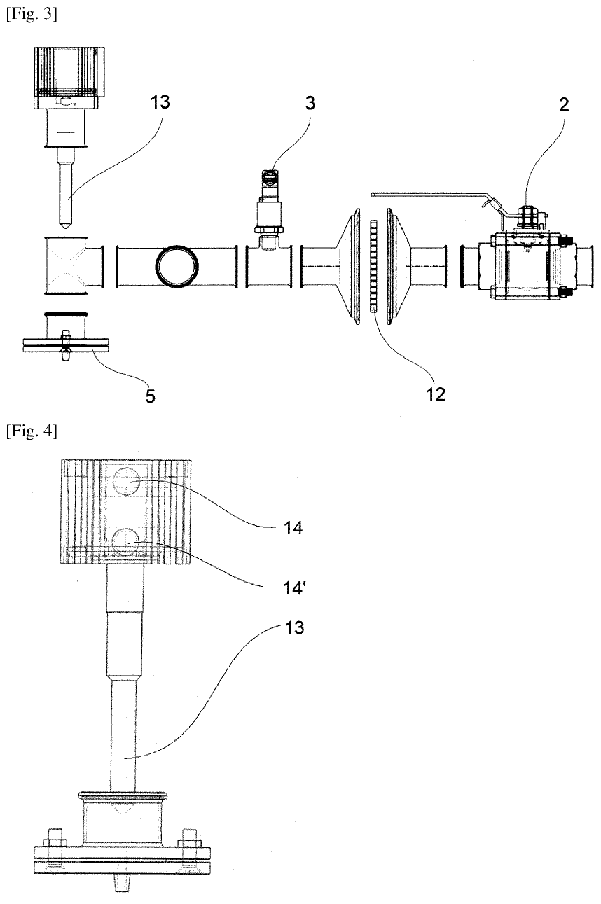

Even if the low viscus material within the container has been pre-filtered, impurities can enter the low viscus material during its delivering. In the embodiment shown in FIG. 2, introducing a mesh screen filter (12) into the pipeline (1) lead impurities injected into a syringe to decrease. A mesh size, namely openings per inch, of the mesh screen filter (12) is depended on size of the impurities and viscosity of the low viscus material, but it is preferably in a range of 50 to 200. It may be located just before the low viscus material is injected into the syringe (6), but the location can be changed. In general, a conventional apparatus for filling a syringe does not have such a filter function.

Furthermore, in the embodiment shown in FIG. 3, it is desirable that the low viscus material-delivering part is designed to be disassembled. This is because it is easy to clean disassembled parts completely after using with any low viscus materials. It helps to the cross contamination between low viscus materials for small scale production of various products.

In the present apparatus, the injection port (5) has a needle valve (4) to open and close the injection port. In the prior art, it is difficult to control precisely the amount of filling and the leak of the low viscus material from the dispensing nozzle of the syringe due to the residual pressure. To solve the problem, the present apparatus is designed to fill the low viscus material with a needle valve (4) that directly opens and closes the injection port (5) to prevent the low viscus material leak caused by the residual pressure.

In FIG. 3, the needle valve (4) is driven by mean of a needle valve cylinder. In the embodiment shown in FIG. 4, it is desirable that the needle valve (4) is connected to a piston rod (13) of the needle valve cylinder. The flowable material is filled in the syringe (6) by vertical reciprocating movement of the piston rod (13). The piston rod (13) is driven by compressed air supplied by an air compressor (not shown) though air supply ports (14) and (14′) of the needle valve cylinder. The needle valve cylinder is known as an air cylinder which uses a power of compressed gas.

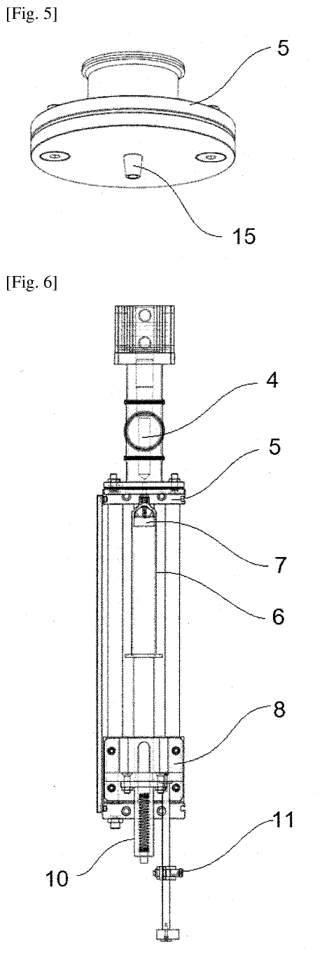

Furthermore, in the embodiment shown in FIG. 5, the injection port (5) may have a syringe filling nozzle (15) to fit the dispensing nozzle of the syringe (6) and to operate semi-automatically cover many different sizes of the syringes (6). A shape and size of the syringe filling nozzle (15) may vary according to the different types of the syringes, but the shape is preferably nipple-like. The syringe filling nozzle (15) can be made of stainless steel, but it can be made of other materials.

FIG. 6 shows a schematic view of a process to fix the syringe (6) under the injection port (5). The injection port (5) is in contact with dispensing nozzle of the syringe (6). The dispensing nozzle is hermetically sealed with respect to the injection port (5).

The syringe fixing plate (8) moves up and down in the longitudinal direction of the syringe to help the syringe (6) to be mounted exactly horizontally on the nipples to avoid the failure of filling the low viscus material. Once the syringe fixing plate (8) abuts a larger aperture of the syringe (5), the syringe fixing plate (8) is stopped. Thus, the syringe fixing plate (8) can fix the syringe (6) to the injection port (5).

The piston (6) is disposed in the syringe (5) to be linearly movable in a longitudinal direction of the syringe, and when the flowable material is injected into the syringe through the injection port (4), the piston (6) hermetically seals the injected low viscus material between the injection port (4) and the piston (6) within the syringe (5). In particular, the piston (6) may be made of a flexible material. The piston (6) is essentially circular when seen from the top along the longitudinal axis. A bottom surface of the piston (6) may be flat or tapered with the lowest point in the middle of the bottom surface, so that the bottom surface has the same incline and can contact essentially evenly with the bottom inner surface of the syringe.

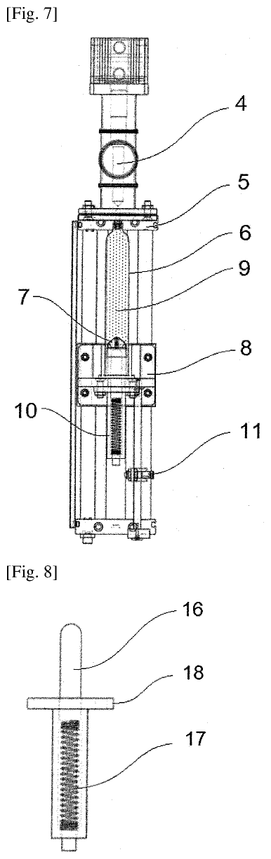

FIG. 7 shows a schematic view of a process to file the low viscus material (9) into the syringe (6). The low viscus material (9) is injected into the syringe (6) fixed the syringe fixing plate (8). The syringe (6) used in the present apparatus may be of any size, but typically has a volume of 3 cc, 5 cc, 10 cc, 30 cc, 55 cc, 100 cc, 250 cc, or 500 cc.

The syringe rod (10) is mounted on the syringe fixing plate (8) to detect the volume of the low viscus material filled into the syringe (5). When the syringe rod (10) comes into contact with the piston (7), and then the piston presses the syringe rod.

The proximity sensor (11) is placed under the syringe fixing plate (8). As shown in FIG. 6, the position of the proximity sensor (11) can be controlled by the volume of the low viscus material filled into the syringe (6) is determined according to the position of the proximity sensor (11). The proximity sensor (11) can detect the contact of the and if the proximity sensor (11) detects the contact of the piston (7), the needle valve (4) of the injection port (5) is closed and the injection of the low viscus material is stopped.

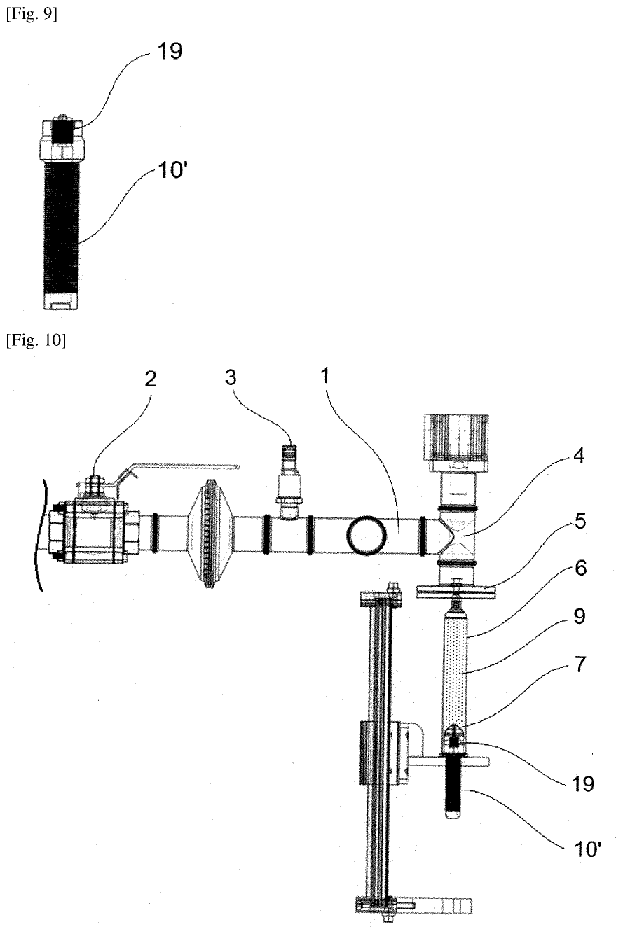

In the embodiment shown in FIG. 8, it is desirable that the syringe rod (10) is composed of a syringe rod (16), a spring (17) to press the rod, and a syringe rod barrel (18) to set a lower end of the spring and to expose an upper end of the rod. The spring can moderately press to the piston through the syringe rod. The syringe rod (16) and the syringe rod barrel (18) can be made of stainless steel, but they can be made of other materials.

In the embodiment shown in FIG. 9, it is desirable that the syringe rod (10′) is composed of syringe rod and a limit sensor (19) mounted on a head of the syringe rod. As shown in FIG. 9, the limit sensor (19) can be controlled by the volume of the low viscus material filled into the syringe (6) is determined according to the contact of the piston (7). That is, if the limit sensor (19) detects the contact of the piston (7), the needle valve (4) of the injection port (5) is closed and the injection of the low viscus material is stopped.

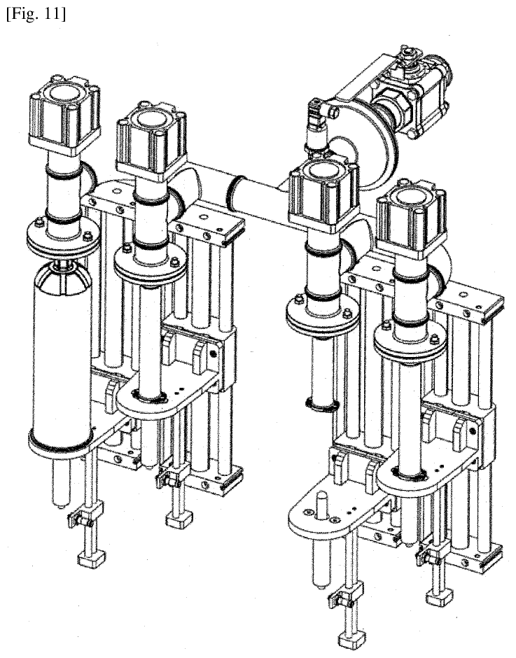

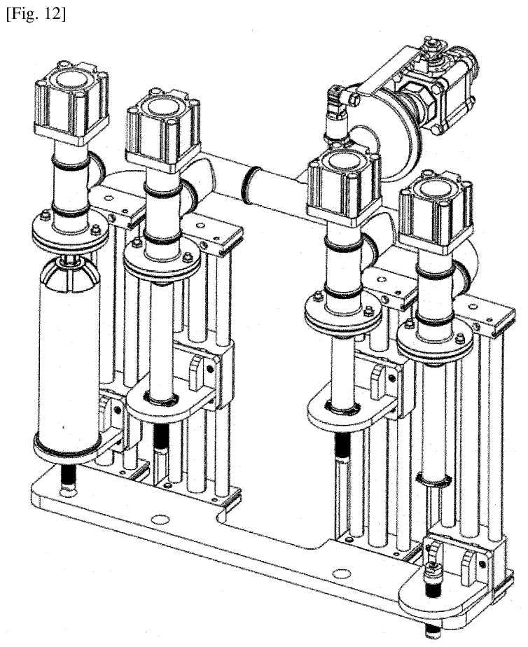

The present apparatus may have one or more injection port. Each injection port may connect the same or different size of the syringes. The conventional apparatuses for filling a syringe are optimized only for mass production of a single product. Therefore, they are limited to different kinds of syringes having different size for various products. However, as shown in FIGS. 11 and 12, according to the present invention, it is possible to increase accuracy of filling syringes with low viscus materials. In the embodiment shown in FIG. 11, wherein the syringe rod is used spring, four separate syringes are located along the injection port (5). While, in the embodiment shown in FIG. 12, wherein the syringe rod mounts limit sensor, four separate syringes are also located along the injection port (5). Each syringe, when empty, is provided with a stopper which locates in the syringe immediately adjacent the syringe fixing plate thereof. The filled syringes are then detached from the injection ports for subsequent usage. According to the present apparatus of FIGS. 11 and 12, it is capable of filling four syringes at the same time. According to the requirement, the number of the injection port to be filled at the same time can be controlled. It is possible manually to operate the syringe fixing plates and the syringe rods, which are operated semi-automatically as well as controlling the pressure supplied with the low viscus material. When the supply of the low viscus material within the container has been exhausted, the injection port (4) may be closed by the needle valve (4) to prevent bubbles forming and the low viscus material leaking.

In embodiments of the present apparatus, it may contain emergency buttons, buttons for filling, and 2 two-hands buttons. It is possible manually to operate the syringe fixing plates and the syringe sensor rods, which are operated semi-automatically as well as controlling the pressure supplied with the flowable material. If the supplied pressure from the container is high, residual pressure could be remained between the valve to the needle valve. Main emergency button can stop injection the flowable material into syringes. Sub-emergency button can stop injecting into each syringe or cartridge. And two hands buttons can be used to prevent possible finger stenosis on the device.

### INDUSTRIAL APPLICABILITY

The present apparatus and method enables rapid filling a low viscus material, and prevents bubbles forming and the material leaking. Therefore, the apparatus and method of the present invention are particularly useful for filling syringes with high viscosity flowable materials such as moisture-curable materials, UV-curable materials, and heat-curable materials, especially moisture-curable materials. Furthermore, the present apparatus and method are useful for mass production of a single product, or small-scale production of various products.

### REFERENCE NUMERALS

1: Pipeline, 2: Valve, 3: Pressure sensor, 4: Needle valve, 5: Injection port, 6: Syringe, 7: Piston, 8: Syringe fixing plate, 9: Low viscus material, 10, 10′: Syringe rod, 11: Proximity sensor, 12: Mesh screen filter, 13: Piston rod, 14, 14′: Air supply, 15: Syringe filling nozzle, 16: Syringe rod, 17: Spring, 18: Syringe rod barrel, 19: Limit sensor

1. An apparatus for filling a syringe, comprising:
a pipeline for delivering a low viscous material discharged from a container;
at least one downwardly directed injection port to connect with a syringe to be filled with the material delivered through the pipeline;
a piston disposed within the syringe to be linearly movable in a longitudinal direction of the syringe and sealing the injected material between the injection port and the piston; and
a syringe rod coupled to the syringe so as to press the piston into the syringe and to indicate a position of the piston;
wherein each injection port has a needle valve to open and close the injection port.

2. The apparatus according to claim 1, wherein the injection port has a syringe filling nozzle.

3. The apparatus according to claim 1, wherein the pipeline is equipped with a mesh screen filter.

4. The apparatus according to claim 1, wherein the pipeline is disassembled for cleaning.

5. The apparatus according to claim 1, wherein the syringe rod is composed of a syringe rod, a spring to press the rod, and a syringe rod barrel to set a lower end of the spring and to expose an upper end of the rod.

6. The apparatus according to claim 1, wherein the syringe rod is composed of a syringe rod, and a limit sensor mounted on a head of the syringe rod.

7. The apparatus according to claim 1, wherein the injection port is opened or closed by the position of the piston.

8. A method for filling a syringe, the method comprising the following steps:
delivering a low viscous material discharged from a container through a pipeline;
filling a syringe connected to a downwardly directed injection port with the material delivered through the pipeline, wherein a piston is disposed within the syringe to be linearly movable in a longitudinal direction of the syringe and sealing the injected material between the injection port and the piston, wherein a syringe rod is coupled to the syringe so as to press the piston into the syringe and indicates a position of the piston; and then
closing the injection port to stop injecting the material by a needle valve of the injection port.

9. The method according to claim 8, wherein the injection port is opened or closed by the position of the piston.
